(19) United States
(12) Patent Application Publication
(10) Pub. No.: US 2011/0086913 A1
(43) Pub. Date: Apr. 14, 2011

(54) METHODS FOR TREATING MYOFASCIAL, MUSCLE, AND/OR BACK PAIN

(76) Inventors: Jie Zhang, Salt Lake City, UT (US); Andrew Crockett, Hampshire (GB); Larry Rigby, Salt Lake City, UT (US)

(21) Appl. No.: 12/752,384

(22) Filed: Apr. 1, 2010

Related U.S. Application Data
(60) Provisional application No. 61/165,600, filed on Apr. 1, 2009.

(51) Int. Cl. A61K 31/24; A61K 31/167; A61P 29/00

(52) U.S. Cl. 514/535; 514/626

(57) ABSTRACT

The present disclosure is drawn to methods for treating myofascial pain, muscle pain, back pain, or combinations of these pains. Specifically, a method for treating myofascial pain, muscle pain, back pain, or combinations thereof includes the application of an analgesic system to a skin surface of a subject experiencing the pain and maintaining the analgesic system on the skin surface for a period of time of at least 30 minutes. The analgesic system applied to the skin surface can include a heating component and a local anesthetic formulation which includes at least one local anesthetic. The heating component can be capable of heating the skin surface to a temperature of 36° C. to 42° C.

This application claims the benefit of U.S. Provisional Patent Application No. 61/165,600, filed Apr. 1, 2009.

### BACKGROUND OF THE INVENTION

Myofascial pain and muscle tension, including back pain, are suffered by many people. The source of these pains can be wide ranging. These pains are frequently treated with non-opioid analgesics delivered orally or by injection. Unfortunately, these treatment options suffer from various drawbacks and undesirable side-effects. These undesirable side-effects are frequently due to their systemic delivery. Accordingly, research continues into alternative methods of ameliorating these pains.

### BRIEF DESCRIPTION OF THE DRAWING

FIG. 1 shows a schematic representation of an exemplary analgesic system in the form of a patch which can be used for treating myofascial, muscle, or back pain.

### DETAILED DESCRIPTION

Before particular embodiments of the present disclosure are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a local anesthetic” includes reference to one or more of such compositions.

The term “skin” includes human skin (intact, diseased, ulcerous, or broken), and mucosal surfaces that are usually at least partially exposed to air such as lips, genital and anal mucosa, and nasal and oral mucosa.

It is also noted that “local anesthetics” in appropriate formulations can be used to provide skin “anesthesia,” which by medical definition means preventing a pain before it happens, such as preventing a pain caused by needle stick. The present disclosure, however, relates to methods of using a local anesthetic formulation to provide “analgesia,” which by medical definition means to reduce or eliminate an existing pain, e.g., myofascial pain, back pain, muscle pain, etc.

The terms “controlled heating” and “controlled heat” are defined as the application of sufficient to heat a skin surface to pre-determined narrow temperature range for a predetermined duration. A controlled heating device that can be used in accordance with systems and methods of the present disclosure can be configured to generate heat promptly when activated. Controlled heating can be achieved through special design of the heating component. For example, controlled heating can be achieved through the use of a properly configured heating element(s) including an exothermic chemical composition. Considerations in generating controlled heat with an exothermic heating component include proper ratios and exothermic chemical compositions used, as well as physical constraints put on the exothermic chemical compositions, e.g., limiting air flow or oxygen contact, spatial configuration of individual heating elements, conductivity of materials used with the exothermic chemical composition, etc. In one embodiment, the heating component can provide heat at a temperature greater than body temperature, but less than a temperature that would cause irreversible skin damage, e.g., burn the skin. An exemplary temperature range that can be implemented for use can be from about 35° C. to about 47° C. In one embodiment, a more preferred temperature range can be from about 36° C. to 42° C. Other desired temperature ranges include from about 38° C. to 42° C. or from 36° C. to 40° C.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein.

As used herein, a plurality of local anesthetics, compounds, and/or heating mechanisms may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0 mm” should be interpreted to include not only the explicitly recited values of about 0.01 mm to about 2.0 mm, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

With this background in mind, the present disclosure provides for methods for treating myofascial pain, muscle pain, back pain, or combinations of these pains. Specifically, a method for treating myofascial pain, muscle pain, back pain, or combinations thereof includes the application of an analgesic system to a skin surface of a subject experiencing the pain and maintaining the analgesic system on the skin surface for a period of time of at least 30 minutes. The application site can be a skin area over the pain, and can be over one or more trigger points. The analgesic system applied to the skin surface can include a heating component and a local anesthetic formulation including at least one local anesthetic. The heating component can be capable of heating the skin surface to a temperature of 36° C. to 42° C. for a sustained period of time within this narrow temperature range.

The methods of the present disclosure can harness the benefits of both increased delivery of the local anesthetic and the therapeutic effect of heating. Furthermore, in some embodiments, the use of heat can actually improve the penetration and benefit of the local anesthetic(s) compared to the use of the same formulation without application of heat. Stated another way, the methods of the present application can provide enhanced transdermal delivery of the local anesthetic through the use of controlled heating, and the added benefit provided by the heat itself. As the skin is heated, the permeability of the skin to the local anesthetics drugs can increase. Additionally, the heating of the skin itself is also believed to reduce the myofascial, muscle, and/or back pain. Accordingly, the combination of the transdermal delivery of the local anesthetic from the local anesthetic formulation with the heat from the heating component can cause a more efficient, i.e. faster and more effective, reduction in pain than either the drug or the heat alone.

As described above, the analgesic systems used in the methods of the present disclosure can comprise two major components: a controlled heating component and a local anesthetic formulation. The local anesthetic formulation can be incorporated in a patch and can include an amount of the local anesthetic to provide, with the help of the heating component, sufficient transdermal delivery of the local anesthetic for reducing or eliminating myofascial, muscle, or back pain. A sufficient transdermal delivery of the local anesthetic is defined as a rate of delivery that is high enough to reduce the pain intensity (as measured by patient report of pain intensity) in an average patient by at least 20%, and preferably at least 30%. The heating device can be configured for application over the patch and the human skin site. Additionally, the heating device can be configured to heat a skin site to which it is applied to a temperature of about 36° C. to about 42° C. The heating device can further be capable of maintaining the skin within the above temperature range for a period of time of at least 30 minutes, or in other embodiments, at least 1 hour.

As stated, the analgesic systems of the present disclosure can include a local anesthetic formulation and a heating component. The local anesthetic formulation can be designed to transdermally deliver at least one local anesthetic. The local anesthetic can generally be any local anesthetic known in the art. In one embodiment, the local anesthetic can be selected from the group consisting of tetracaine, lidocaine, prilocaine, ropivacaine, bupivacaine, benzocaine, and combinations thereof. In another embodiment, the local anesthetic formulation can include lidocaine base. In yet another embodiment, the local anesthetic formulation can include tetracaine base. In yet a further embodiment, the local anesthetic formulation can include a eutectic mixture of lidocaine base and tetracaine base. In another embodiment, the local anesthetic formulation can comprise at least about 30 wt % (in combination) of total local anesthetic, e.g., at least 30 wt % of a 1:1 (by weight) of a eutectic mixture of lidocaine and tetracaine (in combination).

The local anesthetic formulation may also include other ingredients and excipients such as polymers, emulsifiers, chemical permeation enhancers, water or other solvents, and preservatives. In one embodiment, the local anesthetic formulation can include a solidification polymer such as polyvinyl alcohol. In another embodiment, the local anesthetic formulation can include an adhesive polymer which is capable of adhering to skin.

The local anesthetic formulation portion of the analgesic system can have a skin contact region where the local anesthetic formulation contacts the skin surface. The size of the skin contact area can vary depending on the targeted region of the subject's body and the nature of the pain being treated. For example, in on embodiment the skin contact region can have an area of about 2 cm² to about 200 cm². In another embodiment, the skin contact region can have an area of about 3 cm² to about 150 cm². In another embodiment, the skin contact region can have an area of about 5 cm² to about 130 cm². In another embodiment, the skin contact region can have an area of from about 6 cm² to about 100 cm². In another embodiment, the skin contact region can have an area of about 7 cm² to 80 about cm². In another embodiment, the skin contact region can have an area of about 8 cm² to about 40 cm². In yet another embodiment, the skin contact region can have an area between of about 8 cm² to about 13 cm². In one aspect of the disclosure, a layer of adhesive can be coated onto the analgesic outside the contact area between the skin and the local anesthetic formulation for affixing the system on the skin.

The heating components of the analgesic systems used in the method of the present application can be configured to raise the temperature of a skin surface to which the analgesic system is applied to about 36° C. to about 42° C. The heating component can further be configured to maintain the temperature of the skin surface in the above range for a period of time of at least about 30 minutes. In one embodiment, the heating component can be configured to maintain the skin surface in the above described temperature range for a period of at least about 60 minutes.

It is noted that regardless of the duration of heating, the analgesic systems used in the methods of the present disclosure can be configured to relieve myofascial pain, back pain, and/or muscle pain for a period of time beyond the period of time in which the heating component heats and/or which the analgesic system is maintained on the skin surface. In one embodiment, the methods of the present disclosure can provide relief of myofascial pain, back pain, and/or muscle pain for a period of time of at least about 4 hours. In another embodiment, the relief of pain can be for a period of time of at least about 6 hours. In another embodiment, the relief of pain can be for a period of time of at least about 12 hours.

In yet another embodiment, the system can be used on a chronic basis (at least once a day for at least 75% of the days in a period of time lasting at least two weeks). In a further embodiment, the system can be used more than once a day.

The heating components of the analgesic systems used in the methods of the present application can generate heat through a number of mechanisms or means. In one embodiment, the heating component can generate the heat through chemical-based exothermic reactions. Other heating mechanisms can also be used, such as heating by phase transition of supersaturated solutions (such as phase transition of sodium acetate solutions), radiation (microwave or infrared, for example), electricity-resistor means, combinations thereof, and/or other heating sources. In one embodiment, the heating component can be an electric heating device. Such electric heating device can be powered by a variety of sources, for example battery and/or alternating electric current. Electric devices can be configured to provide a predetermined heating profile so that the heating profile is met automatically after engaging or turning on the electric device, e.g., use of timers, programmed electricity supply, finite batter power, etc. Alternatively, the heating profile can be met merely by providing heat at an appropriate temperature with an instruction to the user to remove the heating device after a specific period of time.

In one embodiment, the heating component can generate heat by an exothermic oxidative chemical reaction. The chemical-based exothermic oxidation reaction can generate heat through the contact of the oxidative material, e.g. iron, with ambient air. U.S. Pat. No. 6,756,053, which is incorporated herein by reference in its entirety, describes examples of exothermic heating components and devices.

The amount of exothermic chemical composition in the heating component can vary from depending on the desired duration of heating and the size of the heating component. It can be beneficial to limit the amount of the exothermic chemical composition in the heating component, as a large amount of exothermic chemical composition can cause the heating component to be excessively large or cumbersome and impractical for use. In one aspect, the heating device can include no more than 2 grams of an exothermic chemical composition and can be configured to heat an area of skin greater than about 8 cm².

In addition to the oxidizable component, the exothermic heating composition can further include activated carbon, salt (such as sodium chloride), and water. In one aspect, a water-retaining substance, such as vermiculite or wood powder, can also be included in the heating component.

Depending on the configuration of the heating device, when stored for extended period of time the exothermic heating components can generate gas (believed to be methane and hydrogen) which can cause the packaging in which the exothermic heating component is present to puff up, which in turn can cause complications and problems with respect to storage and transportation. It has been discovered that the inclusion of certain amounts of sulfur-containing compounds, or salts thereof, such as elemental sulfur, sulfates, sulfites, sulfides, or thiosulfates, can reduce or eliminate this gas generation problem when included in the packaging.

Water content in the exothermic chemical composition can have an impact on the heating temperature profile of the heating device. The weight ratio of water to the rest of the ingredients in the exothermic heating component can be in the range of about 1:2.6 to about 1:5.0.

In one aspect, the exothermic chemical composition of the heating component can be manufactured in a manner so as to only have access to ambient oxygen through the holes in a cover that can be made of air-impermeable material. In this way, the flow rate of oxygen from ambient air into the exothermic chemical composition, which in turn can be a factor that can affect the amount and rate of heat generated by the heating component and the temperature of the skin surface on which the analgesic system is applied. Other factors which can influence the temperature and heat generation of the heating component can be the size of the heating component, the amount of the exothermic chemical composition in the heating component, the number and configuration of holes in the heating component's air impermeable cover material, etc.

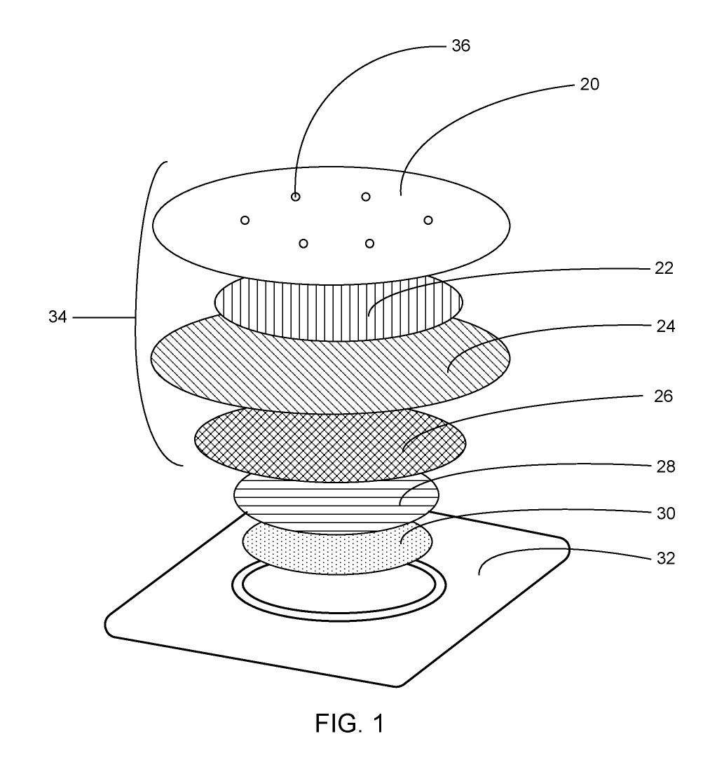

By way of example, FIG. 1 shows a schematic profile of one embodiment of an analgesic system which could be used in accordance with the methods of the present application. The analgesic system includes a heating component 34 and a local anesthetic formulation 30. The heating component includes an air-impermeable top cover film 20 having a plurality of holes 36 therein. When exposed to ambient air, the holes allow for the passage of the ambient air through the air-impermeable top cover film to the exothermic chemical composition 22. The layer of exothermic chemical composition can be disposed between the air-impermeable top cover film and an adhesive film layer 24. The adhesive film layer extends beyond the circumference of the exothermic chemical composition layer and the local anesthetic formulation layer and can function, at least in part, to adhere to the analgesic system to a skin surface. A heat sealable film layer 26 can be below to the adhesive film layer and acts to impede the transfer of substances, particularly moisture, between the local anesthetic formulation layer and the exothermic chemical composition layer. Below the heat sealable film layer, a sodium-borate coated non-woven film layer 28 acts aids in gelling the local anesthetic formulation during manufacturing. The entire analgesic system can be adhered in an air and moisture impermeable packing tray 32 that holds the local anesthetic formulation during storage.

### EXAMPLES

The following examples illustrate the embodiments of the disclosure that are presently best known. However, it is to be understood that the following are only exemplary or illustrative of the application of the principles of the present disclosure. Numerous modifications and alternative compositions, methods, and systems may be devised by those skilled in the art without departing from the spirit and scope of the present disclosure. The appended claims are intended to cover such modifications and arrangements. Thus, while the present disclosure has been described above with particularity, the following examples provide further detail in connection with what are presently deemed to be the most practical and preferred embodiments of the disclosure.

### Example 1

### System for Treating Myofascial, Muscle, and Muscle Pain

A system for treating myofascial, muscle, and muscle pain is prepared having two components: the drug component (drug formulation composition in a patch) and the heating component. Table 1 lists exemplary ingredients in the drug formulation. Table 2 lists exemplary ingredients of the heat generating medium. The drug formulation of the system has a skin contact area of about 10 cm².

TABLE 1

| | Weight | | |
|---|---|---|---|
| | percentage | Amount per | |
| Ingredient | (%) | patch | Function |
| Lidocaine base | 20.00 | 70.00 mg | Active ingredient |
| Tetracaine base | 20.00 | 70.00 mg | Active ingredient |
| Polyvinyl alcohol (PVA) | 7.20 | 25.20 mg | Polymeric matrix |
| Sorbitan monopalmitate | 3.00 | 10.50 mg | Emulsifying agent |
| (Span 40) | | | |
| Purified water | 49.68 | 173.88 mg | Solvent |
| Methyl | 0.10 | 0.35 mg | Preservative |
| parahydroxybenzoate | | | |
| Propylparagydroxybenzoate | 0.02 | 0.07 mg | Preservative |
| Sodium-borate coated | — | 10.84 cm² | Gelling of drug |
| nonwoven film | | | formulation |

TABLE 2

| | Weight Percentage | Weight Per Patch |
|---|---|---|
| COMPONENT | (%) | (grams) |
| Iron powder activated carbon | 50 | 0.80 |
| Activated carbon | 15.63 | 0.25 |
| Sodium chloride | 6.25 | 0.10 |
| Wood flour | 9.38 | 0.15 |
| Water | 18.74 | 0.3 |

The physical configurations of the drug component and the heating component, and their integration, are schematically shown in FIG. 1. The heat generating medium is enclosed in a closed space as shown in FIG. 1, and has access to the external environment only through the 6 holes on the air-impermeable cover. The diameter of each of the holes is about 1/16 of an inch.

### Example 2

### Treating Myofascial Pain

A patient suffering from a myofascial pain in his neck area is treated using an analgesic system as set forth in Example 1. The analgesic system is applied to the skin surface over the pain area and kept there for a period of 2 hours. The intensity of the pain begins to decrease about 30 minutes after application of the analgesic system to the skin surface. After about 1 hour following the commencement of the treatment, the patient begins to feel satisfactory, i.e. at least 20% reduction in pain as measured by the patient report of pain intensity. The pain relief lasts for a period of about 10 hours after the system is removed from the patient's skin.

### Example 3

### Treating Back Pain

A patient suffering from an axial low back pain in his back muscles is treated using a system as described in Example 1. The analgesic system is applied to the skin surface over the pain area and kept there for two hours. The intensity of the pain begins to decrease about 30 minutes after application of the analgesic system to the skin surface. After about one hour following the commencement of the treatment, the patient beings to feel satisfactory pain relief. The pain relief lasts for a period of about 10 hours after the system is removed from the skin area.

### Example 4

### Analgesic System and its Use to Treat Low Back Pain

A system for treating myofascial, muscle, and muscle pain is prepared in a similar manner as described in Example 1 except the system has a larger skin contact area of about 130 cm² and the components of both the drug component and heating components are correspondingly increased. The system is used to treat a patient suffering from axial low-back pain. The analgesic system is applied to the skin surface over the pain area and kept there for two hours. The intensity of the pain begins to decrease about 30 minutes after application of the analgesic system to the skin surface. After about one hour following the commencement of the treatment, the patient begins to feel satisfactory pain relief. The pain relief lasts for a period of about 10 hours after the system is removed from the skin area.

### Example 5

### Analgesic System and its Use to Treat Myofascial Pain

A system for treating myofascial, muscle, and muscle pain is prepared in a similar manner as described in Example 1 except the system has a larger skin contact area of about 80 cm² and the components of both the drug component and heating components are correspondingly increased. The system is used to treat a patient suffering from myofascial pain in his neck. The analgesic system is applied to the skin surface over the pain area and kept there for two hours. The intensity of the pain begins to decrease about 30 minutes after application of the analgesic system to the skin surface. After about one hour following the commencement of the treatment, the patient beings to feel satisfactory pain relief. The pain relief lasts for a period of about 10 hours after the system is removed from the skin area.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A method for treating myofascial pain, muscle pain, back pain, or combination thereof, comprising:
applying an analgesic system to a skin surface of a subject experiencing myofascial pain, muscle pain, back pain, or combination thereof;
maintaining the analgesic system on the skin surface for a period of time of at least 30 minutes;
wherein the analgesic system comprises i) a heating component capable of heating the skin surface to a temperature of 36° C. to 42° C. and ii) a local anesthetic formulation including at least one local anesthetic.

2. The method of claim 1, wherein the local anesthetic formulation includes lidocaine base.

3. The method of claim 1, wherein the local anesthetic includes formulation tetracaine base.

4. The method of claim 1, wherein the local anesthetic is a eutectic mixture of lidocaine base and tetracaine base.

5. The method of claim 1, wherein the local anesthetic is a eutectic mixture of lidocaine base and tetracaine base, and the weight percentage of the eutectic mixture is more than 30% of the weight of the local anesthetic formulation.

6. The method of claim 1, wherein the local anesthetic is selected from the group of tetracaine, lidocaine, prilocaine, ropivacaine, bupivacaine, benzocaine, and combinations thereof.

7. The method of claim 1, wherein the heating component is capable of heating the skin to a temperature of 36° C. to 42° C. for at least 30 minutes.

8. The method of claim 1, wherein the analgesic system is maintained on the skin surface for a period of time of at least 60 minutes.

9. The method of claim 1, wherein the analgesic system is maintained on the skin surface for a period of time of at least two hours.

10. The method of claim 1, wherein the local anesthetic formulation has a skin contact region having an area of about 2 cm² to about 200 cm².

11. The method of claim 1, wherein the local anesthetic formulation has a skin contact region having an area of about 3 cm² to about 150 cm².

12. The method of claim 1, wherein the local anesthetic formulation has a skin contact region having an area of about 5 cm² to about 130 cm².

13. The method of claim 1, wherein the local anesthetic formulation has a skin contact region having an area of about 7 cm² to about 80 cm².

14. The method of claim 1, wherein the local anesthetic formulation has a skin contact region having an area of about 8 cm² and about 40 cm².

15. The method of claim 1, wherein the local anesthetic formulation has a skin contact region, said skin contact region having an area of about 8 cm² to about 13 cm².

16. The method of claim 1, wherein the local anesthetic formulation comprises at least 30% by weight of a eutectic mixture of lidocaine base and tetracaine base and the analgesic system is maintained on the skin for a period of time of at least 30 minutes, and the local anesthetic formulation has a skin contact region having an area of about 8 cm² to about 13 cm².
